(19) United States
(12) Patent Application Publication
(10) Pub. No.: US 2009/0182374 A1
(43) Pub. Date: Jul. 16, 2009

(54) Surgical staple

(76) Inventor: John Keith, Evanston, IL (US)

Correspondence Address: FOLEY & LARDNER LLP, 321 NORTH CLARK STREET, SUITE 2800, CHICAGO, IL, 60654-5313, US

(21) Appl. No.: 12/008,378

(22) Filed: Jan. 10, 2008

(51) Int. Cl. A61B 17/064; A61B 17/068

(52) U.S. Cl. 606/219; 227/175.1

(57) ABSTRACT

A surgical staple for approximating tissue margins. The staple comprises a male portion which includes a first body, a first attachment portion, a male arm and a projection and a female portion separate from the male portion which includes a second attachment portion and a second body arranged to form an opening therein. The projection and the opening are configured to engage with each other and close a wound opening. The staple can be engaged to hold the tissue at varying tensions.

### FIELD OF THE INVENTION

The present invention relates generally to the field of surgical devices.

### BACKGROUND OF THE INVENTION

This section is intended to provide a background or context to the invention that is recited in the claims. The description herein may include concepts that could be pursued, but are not necessarily ones that have been previously conceived or pursued. Therefore, unless otherwise indicated herein, what is described in this section is not prior art to the description and claims in this application and is not admitted to be prior art by inclusion in this section.

A frequently occurring medical condition is open or cut tissues. Tissues such as skin may be cut intentionally, as is done during surgery, or unintentionally, as a result of trauma. This open or cut tissue results in at least one tissue margin, the edge of the tissue adjacent to the opening. In many cases, approximating the tissue margin or bringing the tissue margin closer together enhances the healing process and aids in reducing blood loss. Therefore, devices and methods have been developed to approximate such tissue margins.

A traditional method for closing open or cut tissue is suturing. In suturing, sutures, like stitches, are used to bring the tissue margins together, reducing the size of the opening in the tissue. However, with suturing, approximating the tissue margin can often take more time than is desirable, leaving less time for the practitioner to address other necessary tasks or to see additional patients. Suturing also requires the use of a sharp needle, which can cause injury to the practitioner performing the suturing.

Because of the disadvantages of suturing, surgical staples have been developed and have been used for several years as an alternative to sutures for bringing cut tissue closer together. Existing staples are used to close surgical incisions and to repair traumatic wounds. With existing staples, a more rapid closure of the open tissue can be achieved. Additionally, the use of staples can decrease the likelihood of injury to the physician providing the tissue closure. However, existing staples typically are not as effective as sutures for certain situations in which the tissue opening has an irregular margin, where the tissue opening is non-linear, where the tissue margin is significantly wider than the width of the staple, or where a varying tension is needed on the tissue margins.

### SUMMARY OF THE INVENTION

Various embodiments provide for an improved surgical staple comprising a male part and a female part. Each male and female part have an attachment portion to engage the tissue for approximation. When the male and female parts are engaged, the tissue margin is held in an approximated position. A method according to the present invention allows a person to engage tissue at two locations and approximate and retain the tissue by joining the male and female parts, and an applicator for applying such surgical staples is also provided.

In various embodiments, a surgical staple of the present invention allows the for approximation of irregular, non-linear, and/or wide tissue margins. Various embodiments also allow for approximation of tissue margins under varied tensions. The surgical staple may be used in various surgical procedures including laproscopic surgeries.

These and other advantages and features of the invention, together with the organization and manner of operation thereof, will become apparent from the following detailed description when taken in conjunction with the accompanying drawings, wherein like elements have like numerals throughout the several drawings described below.

### BRIEF DESCRIPTION OF THE DRAWINGS

FIG. 1 is a perspective view of a surgical staple according to an embodiment of the present invention;

FIG. 2 is a perspective view of a surgical staple applicator according to an embodiment of the present invention;

FIG. 3(a) illustrates the insertion of a surgical staple according to an embodiment of the present invention into tissue;

FIG. 3(b) illustrates the surgical staple according to the embodiment of the present invention in FIG. 3(a) approximating tissue at a lower tension;

FIG. 3(c) illustrates the surgical staple according to the embodiment of the present invention in FIG. 3(a) approximating tissue at a higher tension;

FIG. 4 illustrates a top view of a surgical staple according to an embodiment of the present invention;

FIG. 5 illustrates an embodiment of the method for approximating tissue of the present invention;

FIG. 6(a) is a side view of a surgical staple according to an alternative embodiment of the present invention; and

FIG. 6(b) illustrates the surgical staple according to the embodiment shown in FIG. 6(a) after the attachment portions have been bent.

### DETAILED DESCRIPTION OF VARIOUS EMBODIMENTS

Various embodiments provide for an improved surgical staple comprising a male part and a female part. Each male and female part have a attachment portion to engage the tissue for approximation. When the male and female parts are engaged, the tissue margin is held in an approximated position. A method according to the present invention allows a person to engage tissue at two locations and approximate and retain the tissue by joining the male and female parts, and an applicator for applying such surgical staples is also provided.

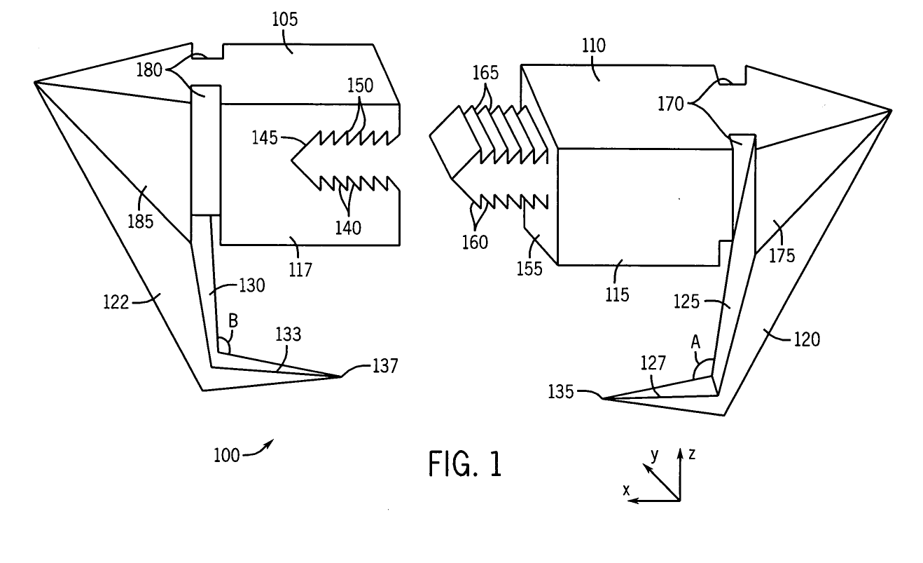

Referring to FIG. 1, a surgical staple according to one embodiment of the present invention, shown generally at 100, comprises a female part 105 and a male part 110 that is separate from the female part 105. The male part 110 includes a first body 115 and a first attachment portion 120. The female part 105 includes a second body 117 and a second attachment portion 122. The first attachment portion 120 includes a first attachment surface 125 and a second attachment surface 127. The second attachment portion 122 includes a third attachment surface 130 and a fourth attachment surface 133. The first attachment surface 125 and the third attachment surface 130 are each designed to transfer a force in the direction of approximation to the tissue to be approximated. The second attachment surface 127 and the fourth attachment surface 133 are each designed to transfer a force from the tissue to the male part 110 and female part 105 respectively in order to retain the staple within the tissue. The second attachment surface 127 terminates in a first attachment portion point 135 with the first attachment portion point 135 used to facilitate the easy insertion of the male part 110 into the tissue. The fourth attachment surface 133 similarly terminates in a second attachment portion point 137 used to facilitate the easy insertion of the female part 105 into the tissue.

In the embodiment shown in FIG. 1, the first and second attachment surfaces 125 and 127 are oriented at an angle A to allow for the easy insertion of the male part 110. Similarly, the third and fourth attachment surfaces 130 and 133 are oriented at an angle B to allow for the easy insertion of the female part 105. In another embodiment, the first and second attachment surfaces 125 and 127 or the third and fourth attachment surfaces 130 and 133 may be a single curved hook surface that allows for the transfer of forces to both approximate the tissue and retain the staple.

Other potential shapes for the first and second attachment portions 120 and 122 may be based on the shape of surgical needles. These additional shapes include, without limitation, round bodied, taper cutting, trocar point, blunt, cutting, and reverse cutting. Different shapes may appropriate for the first and second attachment portions 120 and 122 depending upon the conditions of the procedure at issue.

The female part 105 includes a plurality of openings 140 within a channel 145 that form a plurality of female mating surfaces 150. The male part 110 has a male arm 155 which includes a plurality of projections 160 corresponding to the plurality of openings 140 on the female part and a plurality of male mating surfaces 165. In the embodiment in FIG. 1, the openings 140 comprise notches possessing a triangular shape and the projections comprise tabs possessing a triangular shape, which allows for the male and female parts 110 and 105 to be engaged with each other by bringing the male and female parts together along either an x or y axis. In the embodiment of FIG. 1, the male and female parts, 110 and 105 may be disengaged by sliding the male and female parts, 110 and 105 in opposite directions along the male and female mating surfaces 165 and 150. In other embodiments, the tabs 160 and notches 140 may not be triangular shaped. Instead, the tabs 160 and notches 140 may be cuboidal, ovular, trapezoidal or pyramidal in shape. Also, in yet another embodiment, the male arm 155 may be cylindrically shaped and the channel 145 may be cylindrically shaped to prevent disengagement along any axis.

Varying the distance between the first and third attachment surfaces 125 and 130 will vary the tension on the tissue. To vary the distance between the first and third attachment surfaces 125 and 130, the projections 160 may be engaged in different openings 140 resulting in the ability to vary the tension on the tissue. In the embodiment of FIG. 1 there are 6 projections 160 and 6 openings 140. In other embodiments, the number of projections 160 and openings 140 may be varied as one skilled in the art may contemplate for various applications. Such variations include but are not limited to an embodiment where the channel 145 contains a single opening 140 and the male arm 155 contains a plurality of projections. In another embodiment, the channel 145 may contain a plurality of openings 140 and the male arm 155 may contain a single projection 160.

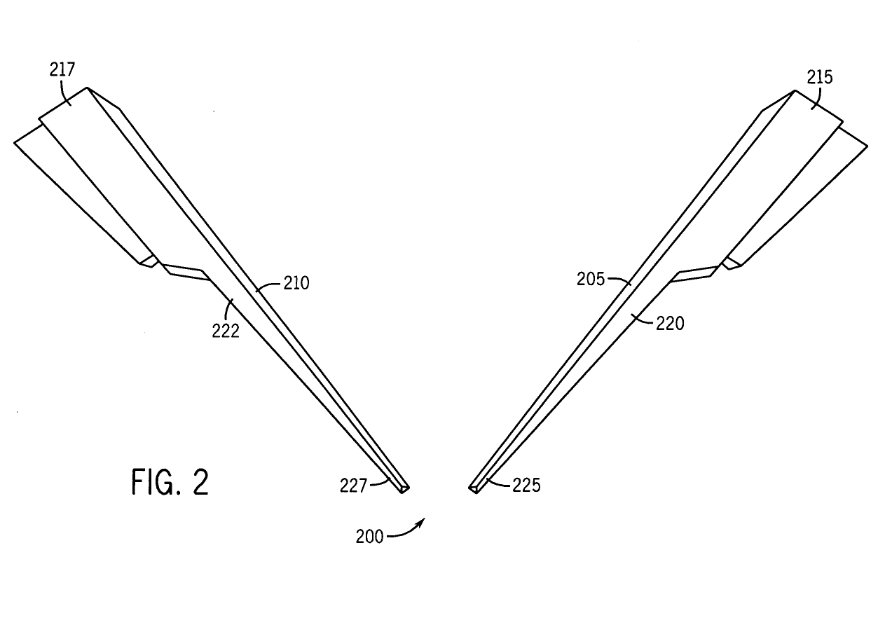

FIG. 1 also shows first applicator engagement channels 170 and a first applicator engagement surface 175 on the male part 110 designed for mating with the staple applicator, illustrated in FIG. 2. The female part 105 contains second applicator engagement channels 180 and a second applicator engagement surface 185 for mating with the staple applicator illustrated in FIG. 2.

FIG. 2 shows a staple applicator generally 200 having a male part applicator 205 and a female part applicator 210. The male part applicator 205 includes a male part applicator handle 215, a male part applicator shaft 220, and a male part engagement section 225. The female part applicator 210 includes a female part applicator handle 217, a female part applicator shaft 222, and a female part applicator engagement section 227.

In another embodiment, the applicator, may automatically dispense the male and female parts 110 and 105.

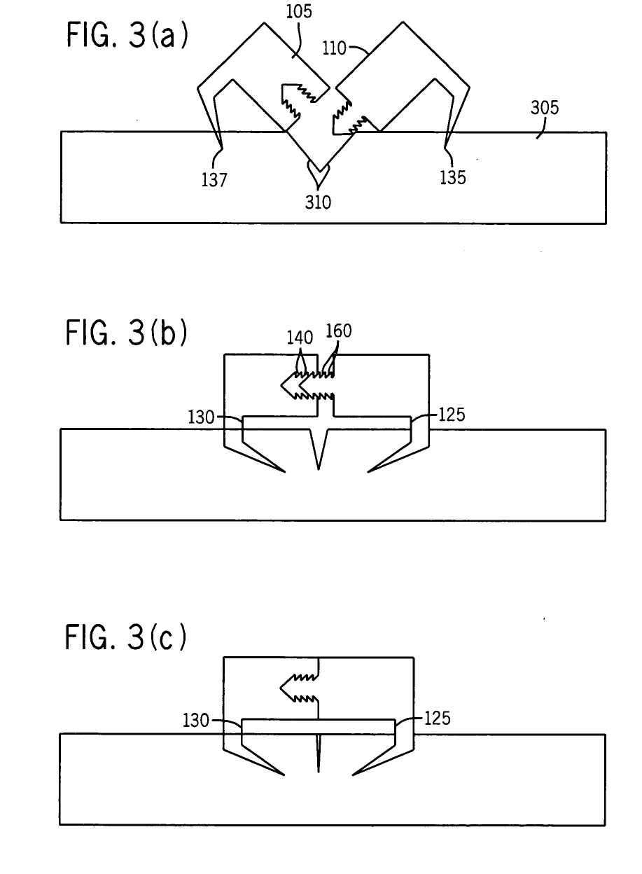

FIG. 3(a) illustrates the insertion of a surgical staple into tissue according to one embodiment of the present invention. The attachment portion points 135 and 137 of the male and female parts 110 and 105 pierce the tissue 305 at locations on the tissue opposite the tissue margins 310. FIG. 3(b) illustrates an embodiment of the invention approximating the tissue margin in a first engagement position. As the male and female parts 110 and 105 are brought closer together towards a second engagement position, as shown in FIG. 3(c), the projections 160 engage with different openings 140. This reduces the total distance between the first and third attachment surfaces 125 and 130 and brings the tissue together under greater tension than would otherwise be possible using a surgical staple having a fixed distance between its first and third attachment surfaces 125 and 130 equal to that of the distance shown in FIG. 3(b).

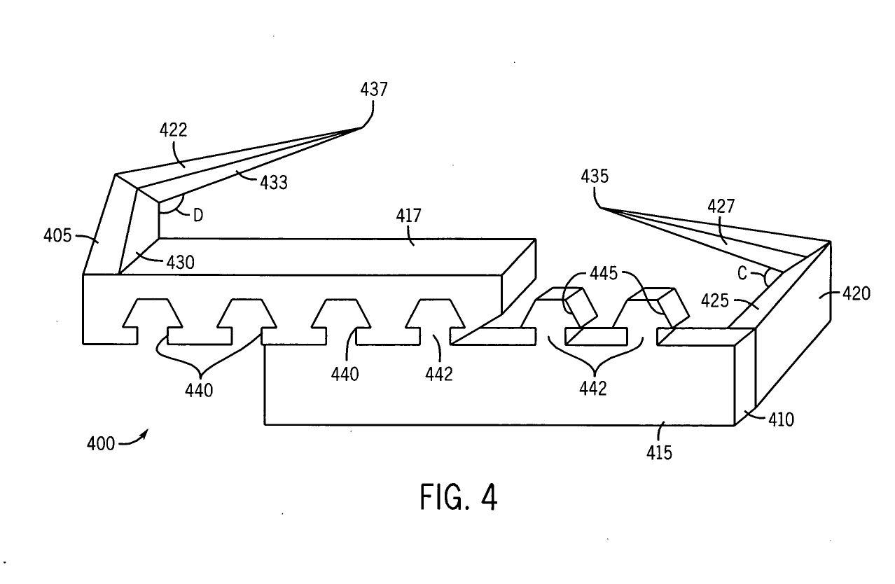

FIG. 4 shows a surgical staple 400 according to another embodiment of the present invention. The surgical staple 400 comprises a female part 405 and a male part 410. The male part 410 includes a first body 415 and a first attachment portion 420. The female part 405 includes a second body 417 and a second attachment portion 422. The first attachment portion 420 includes a first attachment surface 425 and a second attachment surface 427 oriented at an angle C. The second attachment portion 422 includes at third attachment surface 430 and a fourth attachment surface 433 oriented at an angle D. The second and fourth attachment surfaces terminate in a first attachment portion point 435 and a second attachment portion point 437 respectively. A plurality of openings 440 are formed within the second body 417 of the female part 405. A plurality of male arms 442 are located on the first body 415 of the male part 410, having projections 445. As with the embodiment of FIG. 1 described above, the tension on the tissue may be varied by varying the engagement of the projections 445 with the openings 440.

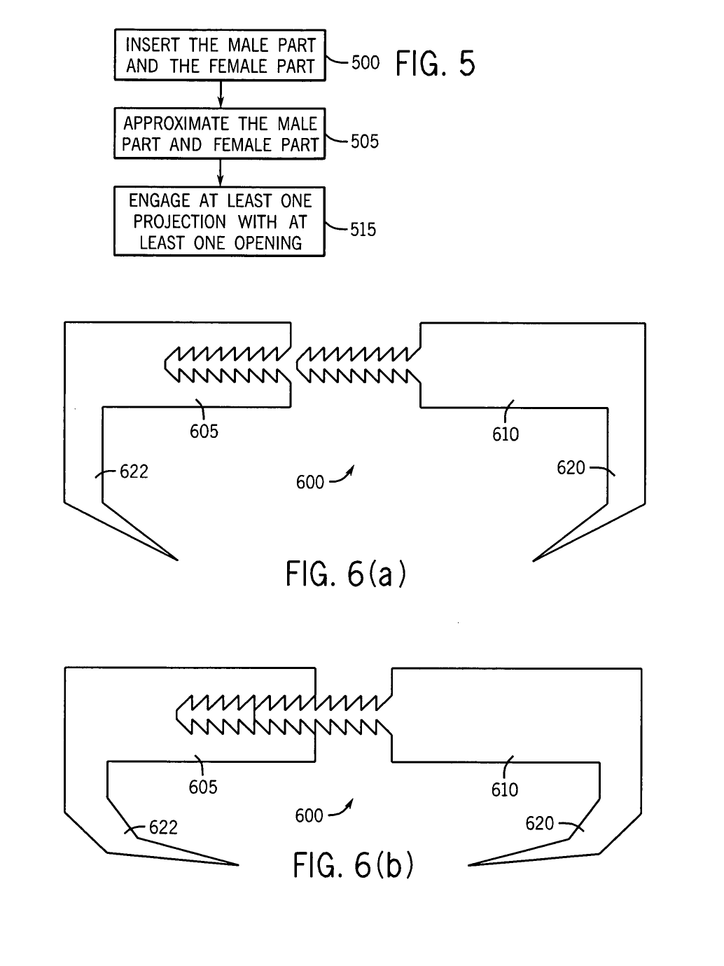

FIG. 5 illustrates an example of a method for approximating tissue according to one particular embodiment of the present invention. At 500 the male and female staple parts are inserted into the tissue. At 505 the male and female staple parts are approximated. At 515 at least one projection is engaged with at least one opening.

FIGS. 6(a) and 6(b) illustrate an additional embodiment of the present invention. The embodiment depicted in FIGS. 6(a) and 6(b) is similar in many respects to the embodiment shown in FIG. 1. In the embodiment shown in FIGS. 6(a) and 6(b), first and second attachment portions 620 and 622 of a surgical staple (shown generally at 600) are bent after insertion of a female part 605 of the second attachment portion and a male part 610 of the first attachment portion 620 into the tissue. This bending provides an ease of insertion of the surgical staple 600 and a more secure hold of the tissue in some situations.

The surgical staples disclosed herein may be designed of various materials allowing the staple to remain in the tissue or may be designed to be removed from the tissue. Therefore, the surgical staples disclosed herein may be made from various types of metal or plastic, combinations of metal and plastic, or synthetic polymers that are designed to be broken down by hydrolysis in tissue fluid depending on whether the staple is to remain in the tissue and be broken down by hydrolysis or removed. Because various embodiments of the surgical staple parts disclosed herein are designed to be brought together through a press-fit, the material for such staples should be sufficiently flexible to allow the projections 160 and 445 and the openings 140 and 440 to deform sufficiently to engage one another as necessary in various embodiments.

The foregoing description of embodiments of the present invention have been presented for purposes of illustration and description. The foregoing description is not intended to be exhaustive or to limit embodiments of the present invention to the precise form disclosed, and modifications and variations are possible in light of the above teachings or may be acquired from practice of various embodiments of the present invention. The embodiments discussed herein were chosen and described in order to explain the principles and the nature of various embodiments of the present invention and its practical application to enable one skilled in the art to utilize the present invention in various embodiments and with various modifications as are suited to the particular use contemplated.

What is claimed is:

1. An apparatus for approximating tissue, comprising:
a male portion including a first body, a first attachment portion, and at least one male arm including at least one projection; and
a female portion separate from the male portion and including a second attachment portion and a second body, the second body arranged to form at least one opening therein,
wherein the at least one projection is configured to selectively mate with one of the at least one opening.

2. The apparatus of claim 1, wherein at least one male arm comprises a single projection, and wherein the at least one opening comprises multiple openings.

3. The apparatus of claim 1, wherein at least one male arm comprises multiple projections, and wherein the at least one channel comprises a single opening.

4. The apparatus of claim 1, wherein first attachment portion comprises a curved hook.

5. The apparatus of claim 1, wherein second attachment portion comprises a curved hook.

6. The apparatus of claim 1, wherein at least a portion of the at least one male arm is oriented substantially perpendicular to the first body, and wherein at least a portion of the at least one channel is oriented substantially perpendicular to the second body.

7. The apparatus of claim 1, wherein at least a portion of the at least one male arm is oriented substantially parallel to the first body and at least a portion of the at least one channel is oriented substantially parallel to the second body.

8. The apparatus of claim 1, wherein the at least one projection comprises at least one triangularly shaped tab, and wherein the at least one opening comprises at least one triangularly shaped notch designed to mate with the at least one triangularly shaped tab.

9. The apparatus of claim 1 wherein the at least one male arm is cylindrically shaped, and wherein the at least one channel is cylindrically shaped.

10. The apparatus of claim 1 wherein the at least one opening is cylindrically shaped, and wherein one of the at least one projection and the at least one male arm is designed to rotate to engage a tab within the cylindrically shaped opening.

11. An apparatus for applying a surgical staple, comprising:
a male part applicator configured to house a male portion of the surgical staple, the male portion including a first body, a first attachment portion, and at least one male arm including at least one projection and
a female part applicator operatively connected to the male part applicator and configured to house a female portion of the surgical staple, the female portion separate from the male portion and including a second attachment portion and a second body, the second body configured to form at least one opening therein,
wherein the at least one projection is configured to selectively mate with one of the at least one opening.

12. The apparatus of claim 11 wherein the at least one male arm comprises a single projection, and wherein the at least one opening comprises multiple openings.

13. The apparatus of claim 11 wherein the at least one male arm comprises multiple projections, and wherein the at least one channel comprises a single opening.

14. The apparatus of claim 11 wherein the first attachment portion comprises a curved hook.

15. The apparatus of claim 11 wherein the second attachment portion comprises a curved hook.

16. A method of approximating tissue using a two piece surgical staple, the method comprising:
inserting into tissue a first attachment portion of a male portion including a first body, the first attachment portion, and at least one male arm including at least one projection;
inserting into the tissue a second attachment portion of a female portion separate from the male portion and including a second body and the second attachment portion, the second body configured to form at least one opening therein,
wherein the at least one projection is configured to selectively mate with one of the at least one opening;
bringing the male portion and female portion together;
engaging the at least one projection with the at least one opening.

17. The method of claim 16 wherein the at least one male arm comprises a single projection, and wherein the at least one opening comprises multiple openings.

18. The method of claim 16 wherein the at least one male arm comprises multiple projections, and wherein the at least one channel comprises a single opening.

19. The method of claim 16 wherein the first attachment portion comprises a curved hook.

20. The method of claim 16 wherein the second attachment portion comprises a curved hook.
